(19) United States
(12) Patent Application Publication
(10) Pub. No.: US 2023/0365969 A1
(43) Pub. Date: Nov. 16, 2023

(54) METHODS FOR INDUCING ADIPOSE TISSUE REMODELING USING RNAI THERAPEUTICS

(71) Applicant: Sirnaomics, Inc., Germantown, MD (US)

(72) Inventor: Michael MOLYNEAUX, Germantown, MD (US)

(21) Appl. No.: 18/355,280

(22) Filed: Jul. 19, 2023

Related U.S. Application Data
(63) Continuation of application No. PCT/US22/80330, filed on Nov. 22, 2022.
(60) Provisional application No. 63/282,174, filed on Nov. 22, 2021.
(60) Provisional application No. 63/282,367, filed on Nov. 23, 2021.

(51) Int. Cl. C12N 15/113; A61P 3/04

(52) U.S. Cl. CPC C12N 15/113; A61P 3/04; C12N 2310/14; B82Y 5/00

(57) ABSTRACT

Methods are provided for inducing the remodeling of adipose tissue in patients to improve health as well as the aesthetic appearance of adipose tissue. RNAi techniques are used with transforming growth factor beta 1 (TGF-β1) and cyclooxygenase 2 (Cox2), both of which have been implicated in disease progression and negative remodeling effects on skin tissues. Administration of two siRNAs targeting TGF-β1 and Cox2 in a single nanoparticle formulation permits entry of the siRNAs into the same cells at the same time and the silencing of target genes in these cells results in favorable remodeling of adipose tissue.

### CROSS REFERENCE TO RELATED PATENT APPLICATIONS

This application is a continuation of PCT/US2022/080330, filed Nov. 22, 2022, and claims the benefit of and priority to U.S. Provisional Patent Application No. 63/282,174, filed Nov. 22, 2021, and U.S. Provisional Patent Application No. 63/282,367, filed Nov. 23, 2021. The contents of each of these applications are incorporated herein by reference in their entireties.

### SEQUENCE LISTING

The instant application contains a Sequence Listing submitted electronically in ST.26 (XML) format and hereby incorporated by reference in its entirety. The XML file, created on Nov. 22, 2022, is named 4690.0085i_Seq List59 and is 73 kilobytes in size.

### FIELD

Methods are provided for inducing the remodeling of adipose tissue in patients to improve health as well as the aesthetic appearance of adipose tissue, including submental, abdominal, thigh (including inner and/or outer thigh), buttock, arm, and breast (including gynecomastia) adipose tissue.

### BACKGROUND

Transforming growth factor 01 (TGF-β1) and cyclooxygenase 2 (Cox2) (also known as prostaglandin-endoperoxide synthase 2 (PTGS2)) have each been implicated in disease progression and negative remodeling effects on skin tissues. TGF-β is upregulated in a number of tumor types and plays a role in stimulating cancer associated fibroblast development. Cox2 upregulation plays a negative role in inducing inflammation and, further, in converting active T-cells to inactive T-reg cells. Previously it has been shown that administration of two siRNAs targeting TGF-β1 and Cox2 in a single nanoparticle formulation permits entry of the siRNAs into the same cells at the same time, and that silencing of target genes in these cells results in antitumoral activity (in patients with in situ squamous cell carcinoma (isSCC)), improved wound healing and the resolution of hypertrophic scars.

### RNAi

RNA interference (RNAi) is a sequence-specific RNA degradation process that provides a relatively easy and direct way to knockdown, or silence, theoretically any gene. In naturally occurring RNA interference, a double stranded RNA is cleaved by an endonuclease into small interfering RNA (siRNA) molecules, overhangs at the 3′ ends. These siRNAs are incorporated into a multicomponent-ribonuclease called RNA-induced-silencing-complex (RISC). One strand of siRNA remains associated with RISC and guides the complex towards a cognate RNA that has sequence complementary to the guider single stranded siRNA (ss-siRNA) in RISC. This siRNA-directed endonuclease digests the RNA, thereby inactivating it. Studies have revealed that the use of chemically synthesized 21-25-nt siRNAs exhibit RNAi effects in mammalian cells, and the thermodynamic stability of siRNA hybridization (at terminals or in the middle) plays a central role in determining the molecule's function.

It is presently not possible to predict with a high degree of confidence which of many possible candidate siRNA sequences potentially targeting a mRNA sequence of a disease gene in fact exhibit effective RNAi activity. Instead, individual specific candidate siRNA polynucleotide or oligonucleotide sequences must be generated and tested in mammalian cell culture to determine whether selective interference with expression of a targeted gene has occurred.

### SUMMARY

Methods are provided for remodeling adipose tissue of a patient by administering an effective amount of a nanoparticle formulation that contains at least one siRNA that targets an mRNA that codes for transforming growth factor beta 1 (TGF-β1) and at least one siRNA that targets an mRNA that codes for cyclooxygenase 2 (Cox2). The formulation also contains a pharmaceutically acceptable carrier. The nanoparticle formulation may contain a pharmaceutically acceptable histidine/lysine copolymer, for example, an HKP and/or HKP(+H). The polymer may be H3K4b. The nanoparticle formulation may be administered via intradermal or subdermal injection, or via intravenous (systemic) administration.

Each siRNA molecule may be, for example, between 19 and 27 nucleotides in length and may be 19 or 25 nucleotides in length. The sense and/or the antisense strand of the siRNA may further contain dTdT overhangs. In a specific embodiment the formulation is STP705.

Also provided are methods of treating a patient with RNAi therapy by administering a nanoparticle formulation to remodel or to sculpt the patient's adipose tissue, where the nanoparticle formulation contains one or more double stranded nucleic acids capable of inhibiting the activity of one or more target genes, and a pharmaceutically acceptable carrier that contains a polypeptide polymer comprising a histidine and a lysine residue. A first target gene may encode transforming growth factor beta 1 (TGF-β1). Another target gene may encode cyclooxygenase 2 (Cox2). The adipose tissue that is remodeled or sculpted may be in a patient suffering from metabolic disruption of adipose tissue. The polypeptide polymer may be HKP or HKP(+H). The double stranded nucleic acid may contain an siRNA, shRNA or miRNA molecule. The siRNA molecule may be conjugated to a non-nucleic acid molecule. Each double stranded nucleic acid contains oligonucleotides, each between 19 and 27 nucleotides in length. Advantageously each strand of the double stranded nucleic acid is 19 nucleotides or 25 nucleotides in length. The formulation may be STP705.

The adipose tissue that may be remodeled and/or sculpted in any of these methods may be, for example, submental, abdominal, thigh (including inner and/or outer thigh), buttock, arm, and/or breast (including gynecomastia) adipose tissue.

### BRIEF DESCRIPTION OF THE DRAWINGS

FIG. 1 shows the gene silencing effects on the target genes as well as the downstream effects on select targets including α-SMA, Col1A1, and Col3A1 following administration of siRNAs in nanoparticle formulation (in the composition known as STP705) comprising HKP. (Zhou et al., infra).

FIG. 2 shows a significant reduction in TGF-β1 protein expression following administration of STP705 to patients.

FIG. 3 shows the reduction in Cox2 protein expression following administration of 10-30 μg of STP705 to patients. For each treatment (results shown in FIGS. 1-3), all patients provided tissue samples, and the protein expression in the matched patient tissue samples was analyzed using immunohistochemistry and semi-quantitatively evaluated by a board-certified MD pathologist.

FIG. 4 shows the sequence of the sense strand of the TGF-β1 and Cox2 siRNAs, along with the sequences of the same genes in various animals. The siRNA sequences for each of TGF-β1 and Cox2 has identity with the genes in humans, mice and monkeys, and only a single nucleotide differs for TGF-β1 in the pig (C-U). The Cox2 siRNA also has identity with the gene sequence in pigs.

FIG. 5 shows the dose-dependent clearance of skin cancer cells from a lesion. The administration of STP705 resulted in little or no scarring on the skin of these patients, particularly relevant from an aesthetic perspective, since many of these lesions occur in exposed regions like the face and neck where scarring from current treatment regimens (surgery, etc.) is commonplace.

FIG. 6 shows 20P-SNC-001 Pig Skins; Mean Histopathology Scores. Group mean standard error of the mean (SEM). Granulomatous inflammation and fibrosis/fibroplasia were seen in all groups receiving test article; scores for both of these features were the highest in the highest STP705 dose group (200 ug/ml). Mean scores were generally lower in animals given TGF-β1 alone siRNA. Findings were also present in the nonsense siRNA controls, so the vehicle may be at least partially responsible for the findings. Superficial dermal/epidermal inflammation and crust formation were very mild and appeared to be associated with animal 2001 rather than a test article administered.

FIG. 7 shows 20P-SNC-001 Pig Skins; Mean Subcuticular Adipose Measurements. Group mean±SEM. Reductions in the thickness of the adipose between the dermis and the most superficial fascial layer were seen with STP-05, TGF-β1 alone siRNA, and NS siRNA; these findings correlated with the presence of fibrosis and inflammation with adipose necrosis in these samples. The overall thickness was much more variable in this study.

FIG. 8 shows Animal 1001, Untreated Area, Site 2 Skin. H&E, 10×. The captured region of untreated skin exhibits normal morphology of the dermis (D) and subcuticular adipose (SC). The most superficial subcuticular fascial plane (black arrowheads) is indicated.

FIG. 9 shows Animal 1001, Area 3, Site B (STP705, 200 μg/ml) Skin. H&E, 10×. The subcuticular adipose (SC) superficial to the fascia (black arrowhead) is narrowed and contains multiple regions of granulomatous inflammation, necrotic adipose (black arrows), and fibrosis (blue arrow). The dermis (D) is indicated.

FIG. 10 shows Animal 1001, Area 4, Site A (NS siRNA Control, Equivalent to 200 μg/ml Dosage) Skin. H&E, 10×. Similar to what was seen in Area 3 (Image 2), there is slight narrowing of the superficial (above the fascia; black arrowhead) subcuticular adipose tissue (SC) with granulomatous inflammation (black arrow) containing an area of fibrosis (blue arrow). The dermis (D) is indicated.

FIG. 11 shows Animal 2001, Untreated Area, Site 2 Skin. H&E, 10×. This region of untreated skin is histologically unremarkable. The dermis (D), subcuticular adipose (SC), and the most superficial fascial layer (black arrowheads) are indicated.

FIG. 12 shows Animal 2001, Area 3, Site A (TGF-β1 alone siRNA, 100 μg/ml), Skin. H&E, 10×. Areas of granulomatous inflammation (black arrows) surrounded by a small amount of increased collagen deposition/fibrosis are present within the superficial subcutis (SC). The superficial fascial layer (black arrowhead) and the dermis (D) are indicated.

FIG. 13 shows Animal 2001, Area 4, Site A (NS siRNA Control, equivalent to 100 μg/ml Dosage) Skin. H&E, 10×. A small area of granulomatous inflammation (black arrow) with fibrosis (blue arrow) is visible within the superficial subcutis (SC), with relatively slight narrowing of this layer observed. The superficial fascia (black arrowhead) and the dermis (D) are indicated.

### DETAILED DESCRIPTION

Methods are provided to remodel or sculpt adipose tissue of a patient by administering an effective amount of an siRNA nanoparticle formulation comprising (i) at least one siRNA or other nucleic acid that inhibits the activity of transforming growth factor beta 1 (TGF-β1) and at least one siRNA or other nucleic acid that inhibits cyclooxygenase (Cox2) (also known as prostaglandin endoperoxide synthase 2 (PTGS2)); and (ii) a polypeptide histidine- and lysine-containing polymer such as HKP and/or HKP(+H). The nanoparticle formulation may be administered locally, e.g., through intradermal injection, or through introduction into the systemic circulation. The oligonucleotide strands of the siRNA or other nucleic acid are advantageously 19-27 nucleotides in length, more advantageously 19 to 25 nucleotides in length.

Methods for remodeling adipose tissue are provided. For example the methods may be used to treat diet-induced metabolic derangement of adipose tissue. The methods comprise administering to a patient at least one siRNA that inhibits the activity of TGF-β1 and at least one siRNA that inhibits Cox2 or PTGS2. Advantageously, the siRNAs are formulated together in a nanoparticle formulation along with a histidine- and lysine-containing polymer, for example, HKP and/or HKP(+H) (see U.S. Pat. Nos. 7,163,695, 7,070,807 and 7,772,201). The formulation may be administered through intradermal, subdermal, or subcutaneous injection or through intravenous (systemic) administration. The methods are advantageously used for remodeling adipose tissue, including submental, abdominal, thigh (including inner and/or outer thigh), buttock, arm, and breast (including gynecomastia) adipose tissue.

TGF-β1 plays an important role in the pathogenesis of obesity, influencing the release of inflammation mediators and promoting remodeling and collagen deposition in the adipose tissue. Sousa-Pinto et al. J. Nutro. Biochem. 38:107-15 (2016). Overfeeding results in a negative remodeling of adipose tissue as deposition is increased and degradation or turnover of the extracellular matrix (“ECM”) is decreased; increased crosslinking and stiffening of ECM fibers is also present. These factors ultimately lead to adipocyte dysfunction, reduced adipogenic capacity and fibrosis. Each one contributes to whole body metabolic dysfunction, e.g., type 2 diabetes and cardiovascular complications. Jones et al. Sci Rep 10:2380 (2020).

Remodeling resulting from over feeding and obesity includes changes in various cell populations of the stromal vascular compartment. In lean subjects, adipose tissue is primarily comprised of M2 macrophages, eosinophils, regulatory T-cells (“Tregs”) and innate lymphoid cells (“ILCs”) that suppress inflammation. Adipose tissue in obese individuals is characterized by an infiltration of B cells and various T cells (i.e., NK and Th1 cells), polarization of M1 macrophages, and a reduction in both Tregs and ILCs, leading to increased inflammation and associated insulin resistance. Proinflammatory cytokines expand profibrotic cells (e.g., ECM-producing cells) and inflammatory immune cells. Id.

We previously have demonstrated that siRNAs that inhibit TGF-β1 and Cox2 expression can induce T-cell penetration into regions where their genes are silenced and the expression of collagen is reduced. Zhou et al. Oncotarget 8(46):80651-80665 (2017). These observations help explain why STP705 at a higher dose (120 μg) resulted in tissue remodeling in patients receiving it. As Jones et al. (2017) demonstrated, adipocytes upregulated several ECM-associated genes in mice after 20 and 34 weeks on a high fat diet, including TGF-β1, inhba, itga5 and ctgf, the collagens (col1a1 and col6a3), elastin (eln), fibronectin (fn1), and other TGF-β family members. Genes associated with Wnt signaling were also upregulated; Wnt signaling has been shown to play a role in dermal fibrosis. Id. TGF-β1 further regulates gene expression through signaling or transcription factor pathways, including SMADs, JNK, ERKs and MRTFA/SRF. MRTFA was implicated as having a role in diet-induced metabolic disruption of adipose tissue by favoring fibrogenesis over adipogenesis. Id.

We demonstrated that co-delivery of the siRNAs simultaneously to the cells resulted in silencing TGFβ1 and Cox2 genes, and human fibroblast apoptosis (Id.). The formulation of HKP into nanoparticles with the siRNAs protected the siRNAs from degradation when administered in vivo and also allowed uptake of the two siRNAs into the same cells at the same time. We showed that HKP mediated siRNA delivery into human hypertrophic scars and the product resulted in a significant reduction in the size of the hypertrophic scars. This was further translated into a reduction of the size of human skin grafts administered to the mice due to an antifibrotic action on the skin samples. The method comprising using the nanoparticle formulation to address wound healing and resolution of scar tissue is disclosed in published US application US20200392507. Examples, without limitation, of both linear and branched histidine and lysine copolymers, that may be used in the disclosed embodiments may be found in U.S. Pat. Nos. 7,163,695, 7,070,807 and 7,772,201, the contents of which are incorporated herein in their entireties.

Tumescent liposuction was developed in 1987 and has since become the gold standard among techniques for subcutaneous adipose tissue removal. Since that time, numerous noninvasive and lipolytic alternatives to liposuction have been developed, including radiofrequency, high intensity focused ultrasound (HIFU), cryolipolysis, nonthermal ultrasound and injection adipolysis. Injection adipolysis is a noninvasive process used to eliminate unwanted adipose tissues in regions of the body. The treatment with the siRNAs for TGF-β1 and Cox2 appear to mimic this effect and may be used as a therapeutic approach to induce the same effect in patients suffering from deleterious adipose tissue remodeling stemming from, e.g., a metabolic derangement.

The disclosed embodiments provide a double stranded (ds) or single stranded (ss) nucleic acid that acts to silence the expression of a gene of interest. In the embodiments disclosed herein the siRNA or other nucleic acid molecules target and bind to complementary sequences on two target genes, TGF-β1 and Cox2, to silence both to elicit the therapeutic effect of remodeling or sculpting adipose tissue that has been previously deleteriously remodeled due to over feeding, obesity and/or other metabolic derangements such as type 2 diabetes and cardiovascular conditions. In some embodiments the siRNA or other nucleic acid molecules are formulated together in a nanoparticle formulation with a polypeptide polymer containing at least one histidine residue and at least one lysine residue. More preferably in some embodiments, the polypeptide polymer is HKP or HKP(+H).

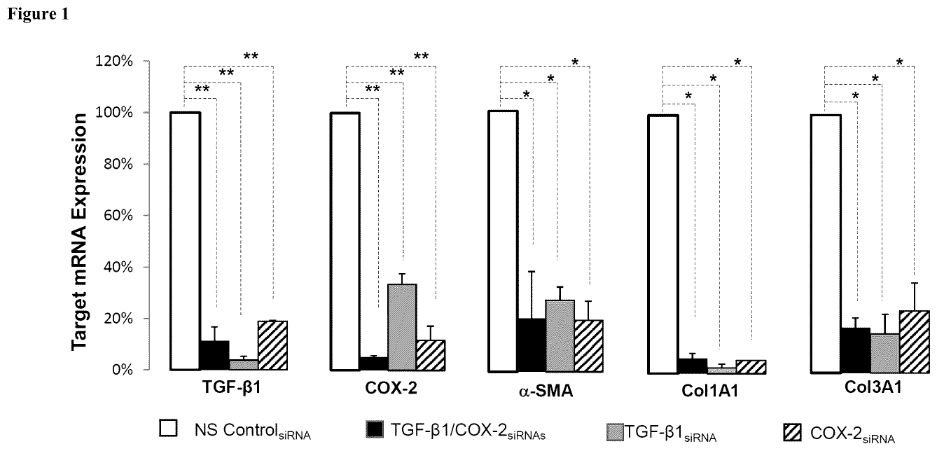

Administration of these siRNAs in a nanoparticle formulation (STP705) comprising HKP results in gene silencing of the targets genes as well as downstream effects on select targets including alphaSMA, Col1A1 and Col3A1 (Zhou et al., supra). See FIG. 1.

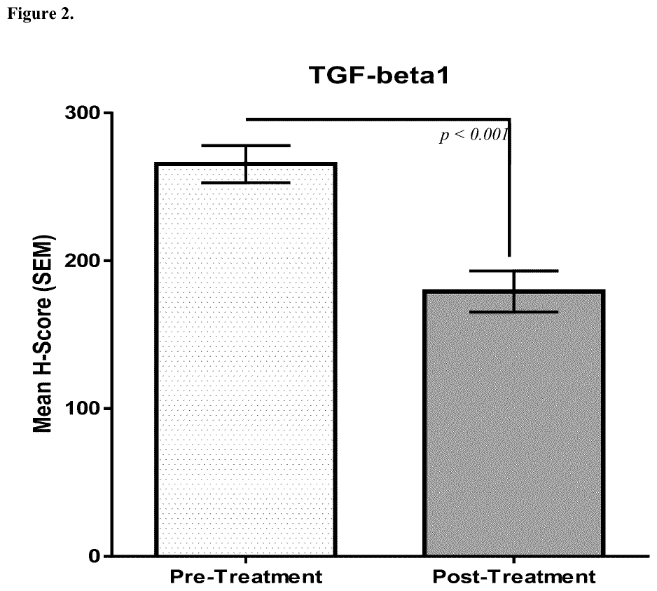

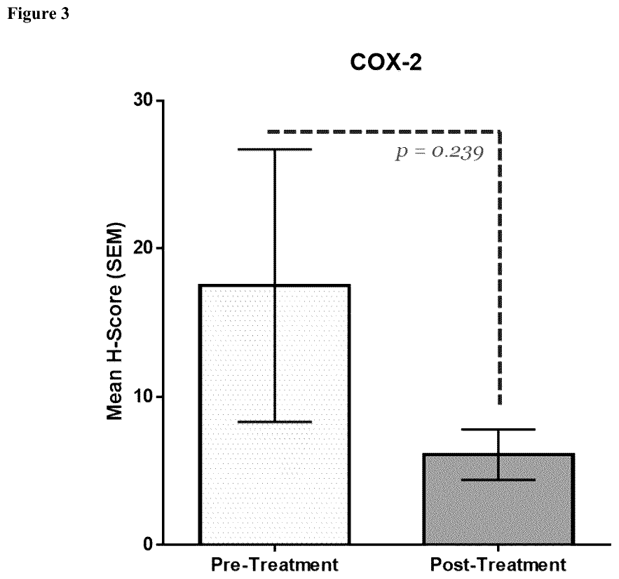

Administration of STP705 in an earlier study to patients with squamous cell carcinoma resulted in a significant reduction in TGF-β1 protein expression as shown in FIG. 2. Administration of 10-30 μg of STP705 to patients also resulted in a reduction in Cox2 protein expression as shown in FIG. 3. Samples of tissue were obtained from all subjects. For each treatment, the protein expression in the matched patient tissue samples was analyzed using immunohistochemistry and semi-quantitatively evaluated by a board-certified MD pathologist.

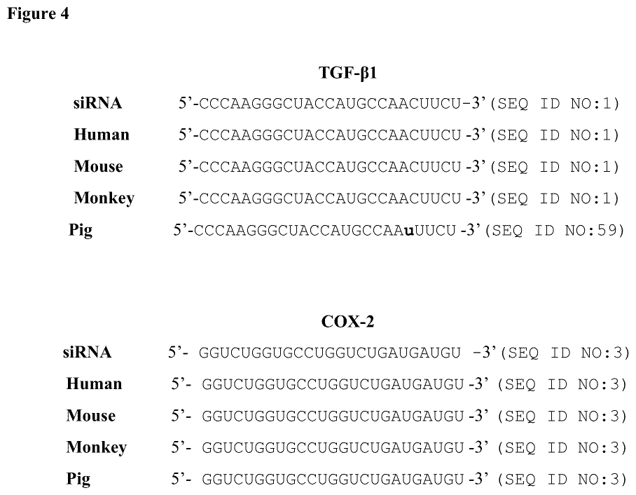

The sequence of the sense strand of the TGF-β1 and Cox2 siRNAs are shown in FIG. 4 along with the sequences of the same genes in various animals. The siRNA sequences for each of TGF-β1 and Cox2 has identity with the genes in humans, mice and monkeys, and only a single nucleotide differs for TGF-β1 in the pig (C-U). The Cox2 siRNA also has identity with the gene sequence in pigs.

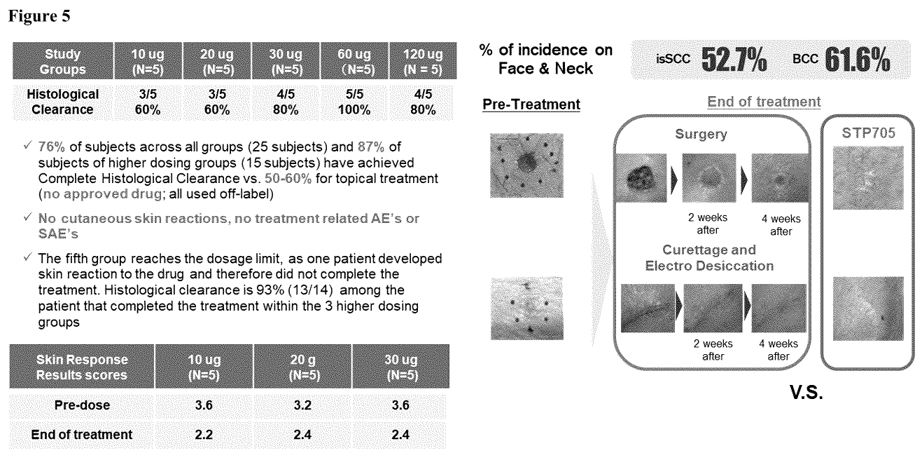

FIG. 5 shows the clearance of skin cancer cells from the lesion in a dose dependent manner. The administration of STP705 resulted in little/no scarring on these patients' skin. This is especially important since many of these lesions occur in exposed regions like the face/neck where scarring from current treatment regimens (surgery or curettage and electro desiccation) is commonplace.

Briefly, 25 subjects were divided equally into five study groups which were treated with 10, 20, 30, 60 and 120 μg respectively of STP705. 76% of subjects across all groups and 87% of subjects of higher dosing groups (15 subjects) achieved Complete Histological Clearance vs. 50-60% for topical treatment. No cutaneous skin reactions, no treatment related adverse events or serious adverse events were reported.

The fifth group reached the dosage limit, as one patient developed skin reaction to the drug and therefore did not complete the treatment. Histological clearance was 93% (13/14) among the patients that completed the treatment within the three higher dosing groups

| | Study Groups | | | | |
|---|---|---|---|---|---|
| | 10 μg | 20 μg | 30 μg | 60 μg | 120 μg |
| | (n = 5) | (n = 5) | (n = 5) | (n = 5) | (n = 5) |
| Histological | 3/5 | 3/5 | 4/5 | 5/5 | 4/5 |
| Clearance | (60%) | (60%) | (80%) | (100%) | (80%) |

| | Skin Response Results scores | | |
|---|---|---|---|
| | 10 μg | 20 μg | 30 μg |
| | (n = 5) | (n = 5) | (n = 5) |
| Pre-dose | 3.6 | 3.2 | 3.6 |
| End of treatment | 2.2 | 2.4 | 2.4 |

The nucleic acid may be a small interfering RNA (siRNA) molecule, comprising a double stranded (duplex) oligonucleotide, wherein the oligonucleotide targets a complementary nucleotide sequence in a single stranded (ss) target RNA molecule. The ss target RNA target molecule is an mRNA encoding at least part of a peptide or protein whose activity promotes inflammation, adipose tissue remodeling or sculpting, wound healing, or scar formation in skin tissue, or it is a micro RNA (miRNA) functioning as a regulatory molecule whose activity promotes inflammation, adipose tissue remodeling or sculpting, wound healing, or scar formation in skin tissue. In one embodiment, the siRNA sequences are prepared in such way that each duplex can target and inhibit the same gene from, at least, both human and mouse, or non-human primates. In certain embodiments, an siRNA molecule binds to an mRNA molecule that encodes at least one protein with 100% or less complementarity. In further embodiments, an siRNA molecule binds to a mRNA molecule that encodes at least one human protein. In still additional embodiments, an siRNA molecule binds to a human mRNA molecule and to a homologous mouse mRNA molecule, i.e., mRNAs in the respective species that encode the same or similar protein.

By “RNA” is meant a molecule comprising at least one, and preferably at least 4, 8 and 12 ribonucleotide residues. The at least 4, 8 or 12 RNA residues may be contiguous. By “ribonucleotide” is meant a nucleotide with a hydroxyl group at the 2′ position of a β-D-ribofuranose moiety. The terms include double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, such as to the end(s) of the dsRNA or internally, for example at one or more nucleotides of the RNA. Nucleotides in the RNA molecules of the disclosed embodiments can also comprise non-standard nucleotides, such as non-naturally occurring nucleotides or chemically synthesized nucleotides or deoxynucleotides. These altered RNAs can be referred to as analogs or analogs of naturally occurring RNA.

As used herein, the term “siRNA” refers to a double stranded nucleic acid in which each strand comprises RNA, RNA analog(s) or RNA and DNA. Typically, the antisense strand of the siRNA is sufficiently complementary with the identified target sequences.

### Preparation of Formulations

In certain embodiments, the disclosed embodiments provide for a pharmaceutical composition comprising the dsRNA agent of the disclosed embodiments. The dsRNA agent sample can be suitably formulated and introduced into the environment of the cell by any means that allows for a sufficient portion of the sample to enter the cell to induce gene silencing, if it is to occur. Many formulations for dsRNA are known in the art and can be used so long as the dsRNA gains entry to the target cells so that it can act. See, e.g., U.S. published patent application Nos. 2004/0203145 A1 and 2005/0054598 A1. For example, the dsRNA agent of the disclosed embodiments can be formulated in buffer solutions such as phosphate buffered saline solutions, liposomes, micellar structures, and capsids. Formulations of dsRNA agent with cationic lipids can be used to facilitate transfection of the dsRNA agent into cells. For example, cationic lipids, such as lipofectin (U.S. Pat. No. 5,705,188), cationic glycerol derivatives, and polycationic molecules, such as polylysine (published PCT International Application WO 97/30731), can be used. Suitable lipids include Oligofectamine, Lipofectamine (Life Technologies), NC388 (Ribozyme Pharmaceuticals, Inc., Boulder, Colo.), or FuGene 6 (Roche) all of which can be used according to the manufacturer's instructions.

Such compositions typically include the nucleic acid molecule and a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in a selected solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It can be appreciated that the method of introducing dsRNA agents into the environment of the cell will depend on the type of cell and the make up of its environment. For example, when the cells are found within a liquid, one preferable formulation is with a lipid formulation such as in lipofectamine and the dsRNA agents can be added directly to the liquid environment of the cells. Lipid formulations can also be administered to animals such as by intravenous, intramuscular, or intraperitoneal injection, or orally or by inhalation or other methods as are known in the art. When the formulation is suitable for administration into animals such as mammals and more specifically humans, the formulation is also pharmaceutically acceptable. Pharmaceutically acceptable formulations for administering oligonucleotides are known and can be used. In some instances, it may be preferable to formulate dsRNA agents in a buffer or saline solution and directly inject the formulated dsRNA agents into cells, as in studies with oocytes. The direct injection of dsRNA agent duplexes may also be done. For suitable methods of introducing dsRNA, see U.S. published patent application No. 2004/0203145 A1.

In one embodiment, the siRNA molecule or other nucleic acid has a length of 19 to 27 base pairs of nucleotides; in another embodiment, the siRNA molecule or other nucleic acid has a length of 20 to 30 base pairs; in still another embodiment the siRNA molecule or other nucleic acid has a length of 24 to 28 base pairs. The molecule can have blunt ends at both ends, or sticky ends at both ends, or one of each. The siRNA molecule may include a chemical modification at the individual nucleotide level or at the oligonucleotide backbone level, or it may have no modifications. In one preferred embodiment an anti-TGF-β1 siRNA or anti-Cox2 siRNA possesses strand lengths of 25 nucleotides. In another, an anti-TGF-β1 siRNA or anti-Cox2 siRNA possesses strand lengths of 19 to 25 nucleotides. In some embodiments, the siRNA molecules can be asymmetric where one strand is shorter than the other (typically by 2 bases e.g. a 21 mer with a 23 mer or a 19 mer with a 21 mer or a 23 mer with a 25 mer). The strands may be modified by inclusion of a dTdT overhang group on the 3′ end of selected strands. An siRNA strand that is 19 nucleotides long advantageously contains an additional dTdT overhang at the 3′ end.

Examples of 25 mer siRNA molecules against TGFB1 and Cox2 include, without limitation, the following:

TABLE 1

| SiRNA SEQ (TGFB and Cox2) | SEQ ID No: |
|---|---|
| cccaagggcu accaugccaa cuucu | 1 |
| agaaguuggc augguagccc uuggg | 2 |
| ggucuggugc cuggucugau gaugu | 3 |
| acaucaucag accaggcacc agacc | 4 |
| gguggcugga acagccagau guguu | 5 |
| aacacaucug gcuguuccag ccacc | 6 |
| gaggagccuu caggauuaca agauu | 7 |
| aaucuuguaa uccugaaggc uccuc | 8 |
| gcugacccug aaguucaucu gcauu | 9 |
| aaugcagaug aacuucaggg ucagc | 10 |
| ggauccacga gcccaagggc uacca | 11 |
| ugguagcccu ugggcucgug gaucc | 12 |
| cccaagggcu accaugccaa cuucu | 13 |
| agaaguuggc augguagccc uuggg | 14 |
| gagcccaagg gcuaccaugc caacu | 15 |
| aguuggcaug guagcccuug ggcuc | 16 |
| gauccacgag cccaagggcu accau | 17 |
| augguagccc uugggcucgu ggauc | 18 |
| cacgagccca agggcuacca ugcca | 19 |
| uggcauggua gcccuugggc ucgug | 20 |
| gaggucaccc gcgugcuaau ggugg | 21 |
| ccaccauuag cacgcgggug accuc | 22 |
| guacaacagc acccgcgacc gggug | 23 |
| cacccggucg cgggugcugu uguac | 24 |
| guggauccac gagcccaagg gcuac | 25 |
| guagcccuug ggcucgugga uccac | 26 |
| ggucuggugc cuggucugau gaugu | 27 |
| acaucaucag accaggcacc agacc | 28 |
| gagcaccauu cuccuugaaa ggacu | 29 |
| aguccuuuca aggagaaugg ugcuc | 30 |
| ccucaauuca gucucucauc ugcaa | 31 |
| uugcagauga gagacugaau ugagg | 32 |
| gauguuugca uucuuugccc agcac | 33 |
| gugcugggca aagaaugcaa acauc | 34 |
| gucuuugguc uggugccugg ucuga | 35 |
| ucagaccagg caccagacca aagac | 36 |
| gugccugguc ugaugaugua ugcca | 37 |
| uggcauacau caucagacca ggcac | 38 |
| caccauucuc cuugaaagga cuuau | 39 |
| auaaguccuu ucaaggagaa uggug | 40 |
| caauucaguc ucucaucugc aauaa | 41 |
| uuauugcaga ugagagacug aauug | 42 |
| gguggcugga acagccagau guguu | 43 |
| aacacaucug gcuguuccag ccacc | 44 |
| gcuggaacag ccagaugugu ugcca | 45 |
| uggcaacaca ucuggcuguu ccagc | 46 |
| ccccagauua ccaucugguu ucaga | 47 |
| ucugaaacca gaugguaauc uggcg | 48 |
| ggagcccggc aauuaugcca ccuug | 49 |
| caagguggca uaauugccgg gcucc | 50 |
| caaggauauc gaaggcuugc uggga | 51 |
| ucccagcaag ccuucgauau ccuug | 52 |
| ggacaagagg cgcaagaucu cggca | 53 |
| ugccgagauc uugcgccucu ugucc | 54 |
| gcaagaucuc ggcagccacc agccu | 55 |
| aggcuggugg cugccgagau cuugc | 56 |
| ccaucugguu ucagaaccgc cgggu | 57 |
| acccggcggu ucugaaacca gaugg | 58 |

Advantageously the composition is STP705. STP705 contains two siRNA oligonucleotide duplexes, with one pair targeting TGF-β1 and one targeting COX-2 mRNA respectively. Each siRNA is double-stranded, 25 nucleotides long, and is blunt ended. The siRNA molecules are formulated with H3K4b (see U.S. Pat. No. 9,642,873)

The sense and antisense strands of the duplex that target TGF-β1 are:

ST-705-1S (Sense):
(SEQ ID NO: 1)
5′ CCCAAGGGCUACCAUGCCAACUUC U 3′
ST-705-1A (Antisense)
(SEQ ID NO: 2)
5′ AGAAGUUGGCAUGGUAGCCCUUGGG 3′

The sense and antisense strands of the duplex that targets COX-2 are:

ST-705-2S (Sense):
(SEQ ID NO: 3)
5′ GGUCUGGUGCCUGGUCUGAUGAUGU 3′
ST-705-2A (Antisense)
(SEQ ID NO: 4)
5′ ACAUCAUCAGACCAGGCACCAGACC 3′

H3K4b is a branched peptide, with a backbone of three L-lysine residues, where the N-terminus and the three lysine ε-amino groups are linked to a histidine-lysine peptide chain with the structure KH₃KH₃KH₃KH₃. The C-terminus of the peptide is amidated.

The molecules are mixed with a pharmaceutically acceptable carrier to provide compositions for administering to a subject. Preferably, the subject is a human. In one embodiment, the composition comprises a pharmaceutically acceptable carrier and at least three siRNA molecules, wherein each siRNA molecule binds an mRNA molecule that encodes a gene selected from the group consisting of pro-inflammatory pathway genes, pro-angiogenesis pathway genes, and pro-cell proliferation pathway genes. In still another embodiment, each siRNA contains at least three siRNA duplexes that target at least three different gene sequences. Preferably, each gene is selected from a different pathway. The disclosed embodiments comprise pharmaceutically effective carriers for enhancing the siRNA delivery into the disease tissues and cells.

In various embodiments of the composition, the carrier comprises one or more components selected from the group consisting of a saline solution, a sugar solution, a polymer, a lipid, a cream, a gel, and a micellar material. Further components or carriers include: a polycationic binding agent, cationic lipid, cationic micelle, cationic polypeptide, hydrophilic polymer grafted polymer, non-natural cationic polymer, cationic polyacetal, hydrophilic polymer grafted polyacetal, ligand functionalized cationic polymer, and ligand functionalized-hydrophilic polymer grafted polymer, biodegradable polyesters, such as poly(lactic acid) (PLA), poly(glycolic acid) (PGA), and poly(lactic-co-glycolic acid) (PLGA), PEG-PEI (polyethylene glycol and polyethylene imine), Poly-Spermine (Spermidine), and polyamidoamine (PAMAM) dendrimers. In preferred embodiments, the carrier is a histidine-lysine copolymer that is believed to form a nanoparticle containing an siRNA molecule, wherein the nanoparticle has a size of about 100 to 400 nm in diameter, or preferably 80 to 200 nm in diameter; and more preferably 80 to 150 nm in diameter. In some embodiments the siRNA molecule may be formulated with methylcellulose gel for topical administration. In some preferred embodiments, the nanoparticle, of a size ranging from 80 to 150, or 80 to 200 nm and containing an siRNA molecule, may be formulated for injection or infusion without methylcellulose gel. Methods of formulating nanoparticles with a methylcellulose gel are known in the art.

The phrase “pharmaceutically acceptable carrier” refers to a carrier for the administration of a therapeutic agent. Exemplary carriers include saline, buffered saline, dextrose, water, glycerol, ethanol, and combinations thereof. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract. The pharmaceutically acceptable carrier of the disclosed dsRNA compositions may be micellar structures, such as a liposomes, capsids, capsoids, polymeric nanocapsules, or polymeric microcapsules.

Polymeric nanocapsules or microcapsules facilitate transport and release of the encapsulated or bound dsRNA into the cell. They include polymeric and monomeric materials, especially including polybutylcyanoacrylate. A summary of materials and fabrication methods has been published (see Kreuter, 1991). The polymeric materials which are formed from monomeric and/or oligomeric precursors in the polymerization/nanoparticle generation step, are per se known from the prior art, as are the molecular weights and molecular weight distribution of the polymeric material which a person skilled in the field of manufacturing nanoparticles may suitably select in accordance with the usual skill.

Modifications and Linkages A dsRNA agent of the disclosed embodiments can be conjugated (e.g., at its 5′ or 3′ terminus of its sense or antisense strand) or unconjugated to another moiety (e.g., a non-nucleic acid moiety such as a peptide), an organic compound (e.g., a dye, cholesterol, or the like). Modifying dsRNA agents in this way may improve cellular uptake or enhance cellular targeting activities of the resulting dsRNA agent derivative as compared to the corresponding unconjugated dsRNA agent, are useful for tracing the dsRNA agent derivative in the cell or improve the stability of the dsRNA agent derivative compared to the corresponding unconjugated dsRNA agent.

As used herein, the term “nucleic acid” refers to deoxyribonucleotides, ribonucleotides, or modified nucleotides, and polymers thereof in single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphorodithioates, phosphoramidates, methyl phosphonates, chiral-methyl phosphonates, 2′-O-methyl ribonucleotides, 2′-Fluoro ribonucleotides, peptide-nucleic acids (PNAs) and unlocked nucleic acids (UNAs; see, e.g., Jensen et al. Nucleic Acids Symposium Series 52: 133-4), and derivatives thereof.

As used herein, “nucleotide” is used as recognized in the art to include those with natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other, see, e.g., Usman and McSwiggen, supra; Eckstein et al., WO 92/07065; Usman et al., WO 93/15187; Uhlman & Peyman, supra, all are hereby incorporated by reference herein). There are several examples of modified nucleic acid bases known in the art as summarized by Limbach, et al, Nucleic Acids Res. 22:2183, 1994. Some of the non-limiting examples of base modifications that can be introduced into nucleic acid molecules include, hypoxanthine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, and others (Burgin, et al., Biochemistry 35:14090, 1996; Uhlman & Peyman, supra). By “modified bases” in this aspect is meant nucleotide bases other than adenine, guanine, cytosine and uracil at 1′ position or their equivalents.

As used herein, “modified nucleotide” refers to a nucleotide that has one or more modifications to the nucleoside, the nucleobase, pentose ring, or phosphate group. For example, modified nucleotides exclude ribonucleotides containing adenosine monophosphate, guanosine monophosphate, uridine monophosphate, and cytidine monophosphate and deoxyribonucleotides containing deoxyadenosine monophosphate, deoxyguanosine monophosphate, deoxythymidine monophosphate, and deoxycytidine monophosphate. Modifications include those naturally occurring that result from modification by enzymes that modify nucleotides, such as methyltransferases. Modified nucleotides also include synthetic or non-naturally occurring nucleotides. Synthetic or non-naturally occurring modifications in nucleotides include those with 2′ modifications, e.g., 2′-methoxy, 2′-methoxyethoxy, 2′-fluoro, 2′-allyl, 2′-O-[2-(methylamino)-2-oxoethyl], 4′-thio, 4′-CH₂—O-2′-bridge, 4′-(CH₂)₂—O-2′-bridge, 2′-LNA or other bicyclic or “bridged” nucleoside analog, and 2′-O—(N-methylcarbamate) or those comprising base analogs.

In connection with 2′-modified nucleotides as described for the present disclosure, by “amino” is meant 2′-NH₂ or 2′-O—NH₂, which can be modified or unmodified. Such modified groups are described, e.g., in Eckstein et al., U.S. Pat. No. 5,672,695 and Matulic-Adamic et al., U.S. Pat. No. 6,248,878. “Modified nucleotides” of the disclosed embodiments can also include nucleotide analogs as described above.

In reference to the nucleic acid molecules of the present disclosure, modifications may exist upon these agents in patterns on one or both strands of the double stranded ribonucleic acid (dsRNA). As used herein, “alternating positions” refers to a pattern where every other nucleotide is a modified nucleotide or there is an unmodified nucleotide (e.g., an unmodified ribonucleotide) between every modified nucleotide over a defined length of a strand of the dsRNA (e.g., 5′-MNMNMN-3′; 3′-MNMNMN-5′; where M is a modified nucleotide and N is an unmodified nucleotide). The modification pattern starts from the first nucleotide position at either the 5′ or 3′ terminus according to a position numbering convention. The pattern of modified nucleotides at alternating positions may run the full length of the strand, but in certain embodiments includes at least 4, 6, 8, 10, 12, 14 nucleotides containing at least 2, 3, 4, 5, 6 or 7 modified nucleotides, respectively. “Alternating pairs of positions” refers to a pattern where two consecutive modified nucleotides are separated by two consecutive unmodified nucleotides over a defined length of a strand of the dsRNA (e.g., 5′-MMNNMMNNMMNN-3′; 3′-MMNNMMNNMMNN-5′; where M is a modified nucleotide and N is an unmodified nucleotide). The modification pattern starts from the first nucleotide position at either the 5′ or 3′ terminus according to a position numbering convention such as those described herein. The pattern of modified nucleotides at alternating positions may run the full length of the strand, but preferably includes at least 8, 12, 16, 20, 24, 28 nucleotides containing at least 4, 6, 8, 10, 12 or 14 modified nucleotides, respectively. It is emphasized that the above modification patterns are exemplary and are not intended as limitations on the scope of the disclosed embodiments.

As used herein, “loop” refers to a structure formed by a single strand of a nucleic acid, in which complementary regions that flank a particular single stranded nucleotide region hybridize in a way that the single stranded nucleotide region between the complementary regions is excluded from duplex formation or Watson-Crick base pairing. A loop is a single stranded nucleotide region of any length. Examples of loops include the unpaired nucleotides present in such structures as hairpins, stem loops, or extended loops.

An anti-TGF-β1 siRNA or anti-Cox2 siRNA advantageously possesses strand lengths of at least 25 nucleotides.

In certain embodiments, the first and second oligonucleotide sequences of the siRNA or other nucleic acid exist on separate oligonucleotide strands that can be and typically are chemically synthesized. In some embodiments, both strands are 25 nucleotides in length, are completely complementary and have blunt ends. In certain embodiments of the disclosed embodiments, the anti-TGF-β1 siRNA or anti-Cox2 siRNA exist on separate RNA oligonucleotides (strands). In certain embodiments TGF-β1 siRNA or anti-Cox2 siRNA agent is comprised of two oligonucleotide strands of differing lengths, with one possessing a blunt end at the 3′ terminus of a first strand (sense strand) and a 3′ overhang at the 3′ terminus of a second strand (antisense strand). The siRNA can also contain one or more deoxyribonucleic acid (DNA) base substitutions.

Suitable siRNA compositions that contain two separate oligonucleotides can be chemically linked outside their annealing region by chemical linking groups. Many suitable chemical linking groups are known in the art and can be used. Suitable groups will not block endonuclease activity on the siRNA and will not interfere with the directed destruction of the RNA transcribed from the target gene. Alternatively, the two separate oligonucleotides can be linked by a third oligonucleotide such that a hairpin structure is produced upon annealing of the two oligonucleotides making up the siRNA composition. The hairpin structure will not block endonuclease activity on the siRNA and will not interfere with the directed destruction of the target RNA.

The dsRNA molecules of the disclosed embodiments are added directly, or can be complexed with lipids (e.g., cationic lipids), packaged within liposomes, or otherwise delivered to target cells or tissues. The nucleic acid or nucleic acid complexes can be locally administered to relevant tissues ex vivo, or in vivo through direct dermal application, transdermal application, or injection, with or without their incorporation in biopolymers.

Dosing

The dsRNA agent can be formulated as a pharmaceutical composition which comprises a pharmacologically effective amount of a dsRNA agent and pharmaceutically acceptable carrier. A pharmacologically or therapeutically effective amount refers to that amount of a dsRNA agent effective to produce the intended pharmacological, therapeutic or preventive result. The phrases “pharmacologically effective amount” and “therapeutically effective amount” or simply “effective amount” refer to that amount of an RNA effective to produce the intended pharmacological, therapeutic or preventive result. For example, if a given clinical treatment is considered effective when there is at least a 20% reduction in a measurable parameter associated with a disease or disorder, a therapeutically effective amount of a drug for the treatment of that disease or disorder is the amount necessary to effect at least a 20% reduction in that parameter.

As defined herein, a therapeutically effective amount of a nucleic acid molecule (i.e., an effective dosage) depends on the nucleic acid selected. For instance, single dose amounts of a dsRNA (or, e.g., a construct(s) encoding for such dsRNA) in the range of approximately 1 μg to up to 10 mg may be administered; in some embodiments, 1, 10, 30, 100, or 1000 μg, or 10, 30, 100, or 1000 ng, or 10, 30, 100, or 1000 μg, may be administered in several areas of the body of a 60 to 120 kg subject. In some embodiments, doses ranging from 60 to 150 μg are administered in this way Advantageously, the compositions are administered subcutaneously, intradermally, or subdermally in multiple areas where remodeling of the adipose tissue is desired, for example, submental, abdominal, thigh (including inner and/or outer thigh), buttock, arm, and breast (including gynecomastia) adipose tissue. Each dose may be, for example, 60-150 μg per cm² administered to several areas in a 60 Kg-120 Kg patient. The skilled artisan will recognize that doses greater or lesser than 60-150 μg per cm² may be administered, for example, between 10-300 μg per cm².

The compositions can be administered from one or more times per day to one or more times per week for the desired length of the treatment; including once every other day. The skilled artisan will appreciate that certain factors may influence the dosage and timing required to effectively treat a subject, including but not limited to the severity of the disease or disorder, previous treatments, the general health and/or age of the subject, and other diseases present. Treatment of a subject with a therapeutically effective amount of a nucleic acid (e.g., dsRNA), protein, polypeptide, or antibody can include a single treatment or, preferably, can include a series of treatments.

In general, a suitable dosage unit of dsRNA will be in the range of 0.00001 to 2 milligrams per kilogram body weight of the recipient per day, or in the range of 0.01 to 20 micrograms per kilogram body weight per day, or in the range of 0.001 to 5 micrograms per kilogram of body weight per day, or in the range of 1 to 500 nanograms per kilogram of body weight per day, or in the range of 0.01 to 10 micrograms per kilogram body weight per day, or in the range of 0.10 to 5 micrograms per kilogram body weight per day, or in the range of 0.1 to 2.5 micrograms per kilogram body weight per day. A pharmaceutical composition comprising the dsRNA can be administered once daily. However, the therapeutic agent may also be dosed in units containing two, three, four, five, six or more sub-doses administered at appropriate intervals throughout the day. In that case, the dsRNA contained in each sub-dose must be correspondingly smaller in order to achieve the total daily dosage unit. The dosage unit can also be compounded for a single dose over several days, e.g., using a conventional sustained release formulation which provides sustained and consistent release of the dsRNA over a several day period. Sustained release formulations are well known in the art. In this embodiment, the dosage unit contains a corresponding multiple of the daily dose. Regardless of the formulation, the pharmaceutical composition must contain dsRNA in a quantity sufficient to inhibit expression of the target gene in the animal or human being treated. The composition can be compounded in such a way that the sum of the multiple units of dsRNA together contain a sufficient dose.

Depending on the particular target gene sequence and the dose of dsRNA agent material delivered, this process may provide partial or complete loss of function for the target gene. A reduction or loss of expression (either target gene expression or encoded polypeptide expression) in at least 50%, 60%, 70%, 80%, 90%, 95% or 99% or more of targeted cells is exemplary. Inhibition of target gene levels or expression refers to the absence (or observable decrease) in the level of target gene or target gene-encoded protein. Specificity refers to the ability to inhibit the target gene without manifest effects on other genes of the cell. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). Inhibition of target target gene sequence(s) by the dsRNA agents of the disclosed embodiments also can be measured based upon the effect of administration of such dsRNA agents upon development/progression of a target gene associated disease or disorder, e.g., deleterious adipose tissue remodeling due to obesity, over feeding or a metabolic derangement, tumor formation, growth, metastasis, etc., either in vivo or in vitro. Treatment and/or reductions in tumor or cancer cell levels can include halting or reduction of growth of tumor or cancer cell levels or reductions of, e.g., 10%, 20%, 30%, 40%, 50%, 60%, 70%, 80%, 90%, 95% or 99% or more, and can also be measured in logarithmic terms, e.g., 10-fold, 100-fold, 1000-fold, 10⁵-fold, 10⁶-fold, 10⁷-fold reduction in cancer cell levels could be achieved via administration of the dsRNA agents of the disclosed embodiments to cells, a tissue, or a subject.

The data obtained from the cell culture assays and animal studies (toxicity, therapeutic efficacy) can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For a compound used in the method of the disclosed embodiments, the therapeutically effective dose can be estimated initially from cell culture assays. A dose may be formulated in animal models to achieve a circulating plasma concentration range that includes the IC.sub.50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Administration

Suitably formulated compositions of the disclosed embodiments can be administered by means known in the art such as by parenteral routes, including intravenous, intramuscular, intraperitoneal, subcutaneous, intradermal, subdermal, transdermal, airway (aerosol), rectal, vaginal and topical (including buccal and sublingual) administration. In some embodiments, the pharmaceutical compositions are administered by intravenous or intraparenteral infusion or injection.

A formulation is prepared to be compatible with its intended route of administration. Examples of routes of administration include parenteral, e.g., intravenous, intradermal, subcutaneous, subdermal, oral (e.g., inhalation), transdermal (topical), transmucosal, and rectal administration. Solutions or suspensions used for parenteral, intradermal, subdermal or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The siRNA formulations can also be administered by transfection or infection using methods known in the art, including but not limited to the methods described in McCaffrey et al. (2002), Nature, 418(6893), 38-9 (hydrodynamic transfection); Xia et al. (2002), Nature Biotechnol., 20(10), 1006-10 (viral-mediated delivery); or Putnam (1996), Am. J. Health Syst. Pharm. 53(2), 151-160, erratum at Am. J. Health Syst. Pharm. 53(3), 325 (1996).

Further, the siRNA formulations can also be administered by a method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al. (1998), Clin. Immunol. Immunopathol., 88(2), 205-10. Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

### Treatments

The presently disclosed embodiments provides for both prophylactic and therapeutic methods of treating a subject at risk of (or susceptible to) a disease, disorder or condition caused or exacerbated, in whole or in part, by TGF-β1 and/or Cox2 gene expression.

“Treatment”, or “treating” as used herein, is defined as the application or administration of a therapeutic agent (e.g., a dsRNA agent or vector or transgene encoding same) to a patient, or application or administration of a therapeutic agent to an isolated tissue or cell line from a patient, who has the disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

In one aspect, the disclosed embodiments provide a method for preventing in a subject, a disease or disorder as described above (including, e.g., prevention of the commencement of transforming events within a subject via inhibition of TGF-β1 and Cox2 expression), by administering to the subject a therapeutic agent (e.g., a dsRNA agent or vector or transgene encoding same). Subjects at risk for the disease can be identified by, for example, one or a combination of diagnostic or prognostic assays as described herein. Administration of a prophylactic agent can occur prior to the detection of, e.g., cancer in a subject, or the manifestation of symptoms characteristic of the disease or disorder, such that the disease or disorder is prevented or, alternatively, delayed in its progression.

The dsRNA molecules (siRNA) can be used in combination with other treatments to treat, inhibit, reduce, or prevent a deleterious adipose remodeling in a subject or organism.

Another aspect of the disclosed embodiments pertains to methods of treating subjects therapeutically, i.e., altering the onset of symptoms of the disease or disorder. These methods can be performed in vitro (e.g., by culturing the cell with the dsRNA agent) or, alternatively, in vivo (e.g., by administering the dsRNA agent to a subject).

With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the disclosed embodiments provides methods for tailoring an individual's prophylactic or therapeutic treatment with either the target TGF-β1 and Cox2 genes or modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

Therapeutic agents can be tested in a selected animal model. For example, a dsRNA agent (or expression vector or transgene encoding same) as described herein can be used in an animal model to determine the efficacy, toxicity, or side effects of treatment with said agent. Alternatively, an agent (e.g., a therapeutic agent) can be used in an animal model to determine the mechanism of action of such an agent.

In human patients the efficacy of the treatment in, for example, submental tissue, can be determined using the Clinician-Reported Submental Fat Rating Scale (CR-SMFRS)(see McDiarmid et al, Aesth Plast Surg (2014) 38:849-860).

The disclosed embodiments described and claimed are not to be limited in scope by the specific preferred embodiments referenced herein, since these embodiments are intended as illustrations, not limitations. Any equivalent embodiments are intended to be within the scope of this disclosure, and the embodiments disclosed are not mutually exclusive. Indeed, various modifications to the embodiments, in addition to those shown and described herein will become apparent to those skilled in the art from the foregoing description. Such modifications are also intended to fall within the scope of the appended claims.

The terms and words used in the following description and claims are not limited to conventional definitions but, rather, are used to enable a clear and consistent understanding of the disclosure. Accordingly, it should be apparent to those skilled in the art that the description of various embodiments is provided for illustration purpose only and not for the purpose of limiting the disclosure with respect to the appended claims and their equivalents.

It is to be understood that the singular forms “a,” “an,” and “the” include the plural forms unless the context clearly dictates otherwise, e.g., reference to “a dermatologically active compound” includes reference to one or more such compounds.

Unless otherwise defined herein, all terms used have the same meaning as commonly understood by a person of ordinary skill in the art. Terms used herein should be interpreted as having meanings consistent with their meanings in the context of the relevant art.

As used herein, the terms “comprising,” “comprise” or “comprised,” in reference to defined or described elements of any item, composition, formulation, apparatus, method, process, system, etc., are intended to be inclusive or open ended, and includes those specified elements or their equivalents. Other elements can be included and still fall within the scope or definition of the defined item, composition, etc.

The term “about” or “approximately” means within an acceptable error range for the particular value as viewed by one of ordinary skill in the art; this depends in part on how the value is measured or determined based on the limitations of the measurement system.

“Co-administer” or “co-deliver” refers to the simultaneous administration of two pharmaceutical formulations in the blood or other fluid of an individual using the same or different modes of administration. Pharmaceutical formulations can be concurrently or sequentially administered in the same pharmaceutical carrier or in different ones.

The terms “subject,” “patient,” and “individual” are used interchangeably.

### Example 1

Two patients with in situ Squamous Cell Carcinoma (isSCC) were being treated in a clinical trial with the nanoparticle formulation administered directly into the tumors at doses of 10, 20, 30, 60 or 120 μg each. Six doses were administered per tumor on a weekly basis. Clinical results of this trial demonstrated a significant dose-dependent effect of the treatment in reducing the volume of tumor and, at doses of 30 μg, 60 μg, and 120 μg per dose, clinical clearance of the lesions in 13 of 15 (87%) of patients with the tumors was observed.

IHC staining of samples recovered from biopsies of the tumors post administration of the compound suggested that increased efficacy through dual administration of TGF-β1 and COX-2 siRNAs in the same formulation resulted in an increase in recruitment of CD4+ and CD8+ T-cells into the solid tumor. This effect is augmented by reducing the TGF-β gradient that occurs surrounding tumors and that has been demonstrated to inhibit penetration of T-cells into the tumors (Tauriello et al., Nature 554:538-546 (2018); Mariathasan et al., Nature 554:544-548 (2018)). Elevated Cox2 also plays a role in inhibiting active T-cell recruitment to tumors (Gao et al., Digestion 79:169-76 (2009)). Inhibition of Cox2 expression within the tumor microenvironment is expected to inhibit the conversion of active T-cells into Tregs (Id.)—augmenting the activity of the recruited T-cells. Therefore, the combination treatment has a surprising and dramatic effect in recruiting T-cells and maintaining their ability to fight against non-self cells (e.g., tumor cells).

Surprisingly, at the highest dose (120 μg) of STP705 two patients exhibited skin changes (a slight elevation in the skin in the area of treatment), findings consistent with panniculitis, an inflammation of subcutaneous adipose tissue. Both the dermal and epidermal layers examined from these patients did not show signs of inflammation or of the effect seen in the subcutaneous adipose tissue. This panniculitis indicates reduction of adipose tissue in the region. This remodeling effect of treatment with the nanoparticle formulation results in changes in adipocyte content and distribution and improves the esthetic appearance of excess adipose tissue, including submental, abdominal, thigh (including inner and/or outer thigh), buttock, arm, and breast (including gynecomastia) adipose tissue.

Taken together, these observations and data support the utility of administering siRNAs against TGF-β1 and Cox2 in a single nanoparticle delivery system. Both siRNAs were delivered intradermally in the same formulation to the same cells at the same time, which induced the necessary changes in the skin to cause adipose remodeling/sculpting.

### Example 2

### Methods

Thirty-six formalin-fixed skin samples including 30 full thickness samples and 6 punch biopsies from two male Yucatan minipigs were used. Pre-dosing biopsies were collected, and animals received test articles by subdermal injection on Days 0, 7, and 14; animals were sacrificed on Day 56. One slide per block was sectioned and stained with hematoxylin and eosin (H&E). Glass slides were evaluated by a board-certified veterinary pathologist using light microscopy. Histologic lesions were graded for severity (0=absent; 1=minimal; 2=mild; 3=moderate; 4=marked; 5=severe). Glass slides were also scanned using an Aperio AT2 slide scanner for the creation of whole slide images; measurements were performed using Aperio ImageScope calibrated software. Subcuticular adipose thickness (μm) was measured in 10 sites across each sample. Five sites were measured between the deep aspect of the dermis to the most superficial fascial plane and five sites were measured between the deep aspect of the dermis to the skeletal muscle/deepest portion of adipose tissue captured on the slide.

### Results

Morphologic Pathology (H&E)

Findings associated with the administration of either test article were granulomatous inflammation with fat necrosis in the subcutis and fibrosis/fibroplasia in the subcutis. Granulomatous inflammation was characterized by the infiltration/aggregation of macrophages, multinucleated giant cells, low numbers of neutrophils, and lymphocytes which often formed tight aggregates. Inflammatory cells often encircled necrotic adipocytes with variable degrees of mineralization observed. Fibrosis/fibroplasia was characterized by the replacement of adipocytes by generally loosely arranged fibrous connective tissue/collagen with scattered fibroblasts, capillary profiles, and a few infiltrating inflammatory cells. Both findings were seen primarily within the superficial subcutis, superficial to the first fascial plane layer.

These findings were seen in sites receiving either STP705, TGF-β1 alone siRNA, or the nonsense siRNA control articles but not those from untreated areas or pre-dose biopsies. Thus, these findings may be associated with the vehicle, rather than specific test articles. However, a loose association was seen with the dose of test article administered (FIG. 1).

Other findings, including dermal inflammatory cell infiltration and serocellular crusts on the epidermal surface, were sporadically present and represent the variability in typical background cellular infiltrates in the skin of pigs.

Subcuticular Thickness Measurements

Due to the variability in sampled structures (such as skeletal muscle) and the varied thickness of samples, measurements were performed in two zones, between the deep dermal border and the most superficial fascial layer, and between the deep dermal border and either the deep skeletal muscle border or the deep tissue margin of the tissue. The full thickness of the subcuticular adipose tissue varied considerably among sampled sites and treatments (FIG. 2); however, decreased subcuticular thickness (up to the superficial fascia) was reduced in test article-treated samples compared to untreated or pre-dosing samples. This reduced subcuticular thickness corresponded with the presence of inflammation and fibroplasia in this zone.

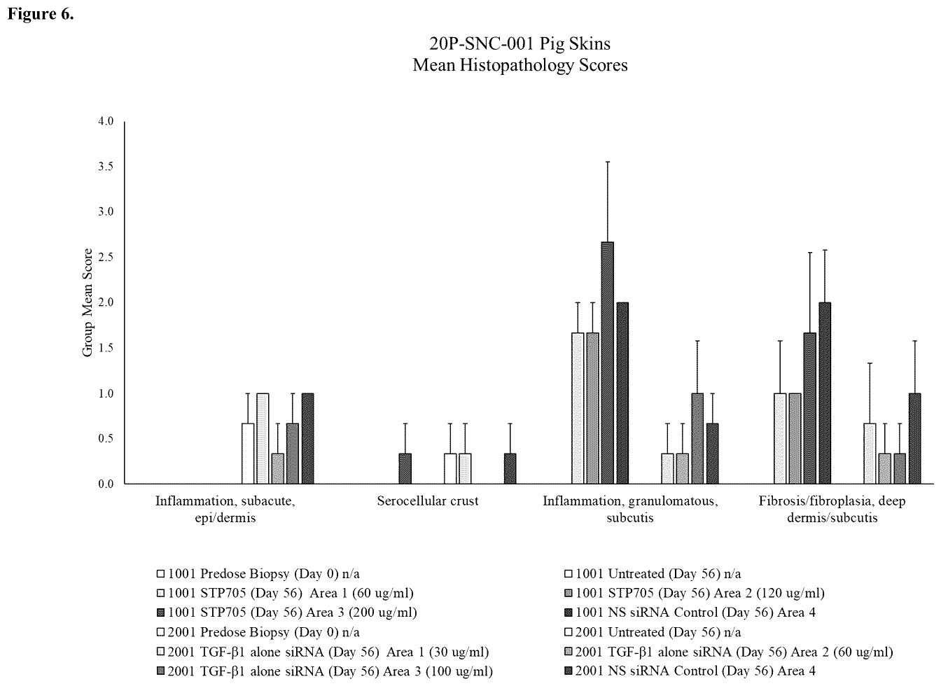

FIG. 6 shows 20P-SNC-001 Pig Skins; Mean Histopathology Scores. Group mean±standard error of the mean (SEM). Granulomatous inflammation and fibrosis/fibroplasia were seen in all groups receiving test article; scores for both of these features were the highest in the highest STP705 dose group (200 ug/ml). Mean scores were generally lower in animals given TGF-β1 alone siRNA. Findings were also present in the nonsense siRNA controls, so the vehicle may be at least partially responsible for the findings. Superficial dermal/epidermal inflammation and crust formation were very mild and appeared to be associated with animal 2001 rather than a test article administered.

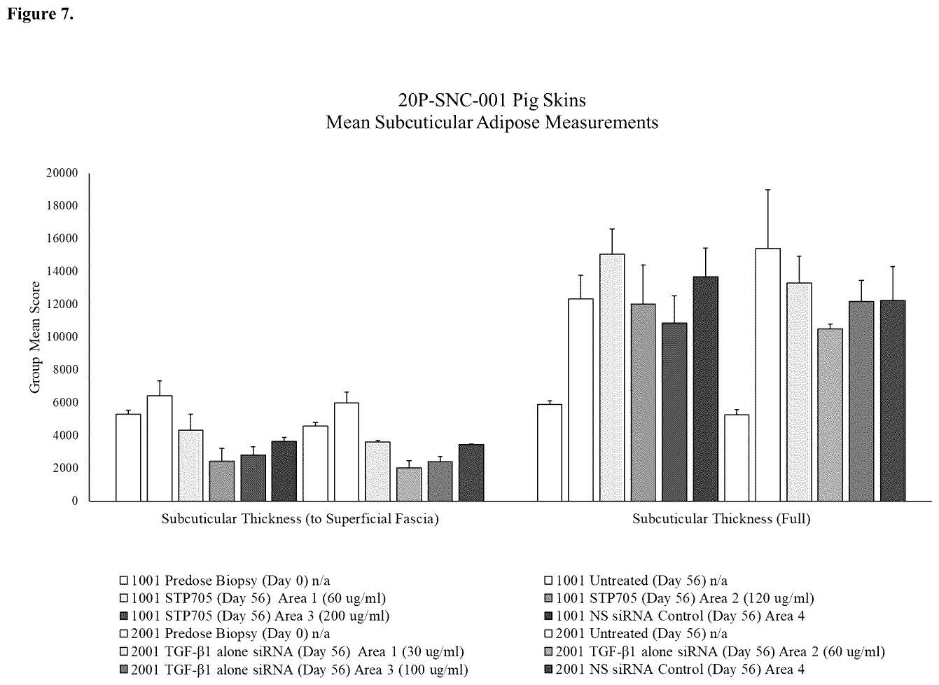

FIG. 7 shows 20P-SNC-001 Pig Skins; Mean Subcuticular Adipose Measurements. Group mean±SEM. Reductions in the thickness of the adipose between the dermis and the most superficial fascial layer were seen with STP-05, TGF-β1 alone siRNA, and NS siRNA; these findings correlated with the presence of fibrosis and inflammation with adipose necrosis in these samples. The overall thickness was much more variable in this study.

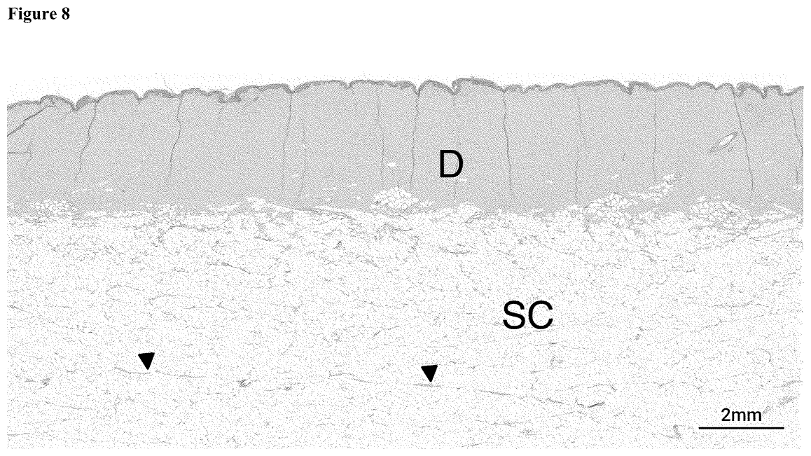

FIGS. 8-13 show representative photomicrographs.

FIG. 8 shows Animal 1001, Untreated Area, Site 2 Skin. H&E, 10×. The captured region of untreated skin exhibits normal morphology of the dermis (D) and subcuticular adipose (SC). The most superficial subcuticular fascial plane (black arrowheads) is indicated.

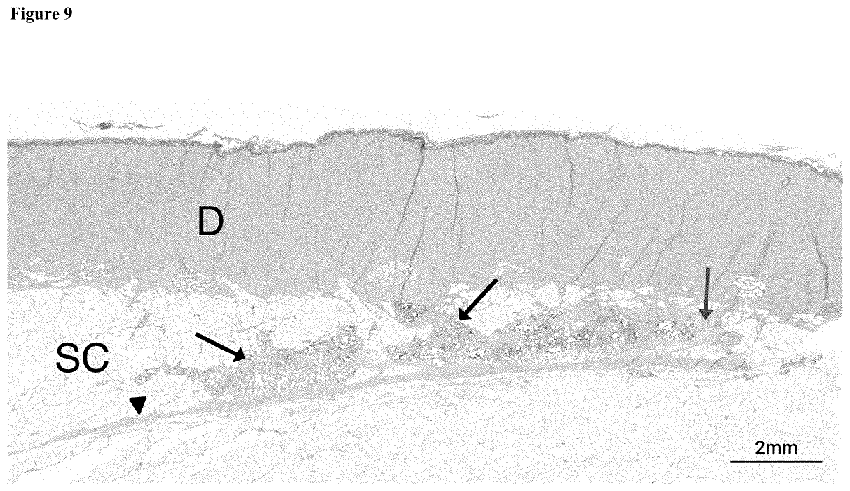

FIG. 9 shows Animal 1001, Area 3, Site B (STP705, 200 μg/ml) Skin. H&E, 10×. The subcuticular adipose (SC) superficial to the fascia (black arrowhead) is narrowed and contains multiple regions of granulomatous inflammation, necrotic adipose (black arrows), and fibrosis (blue arrow). The dermis (D) is indicated.

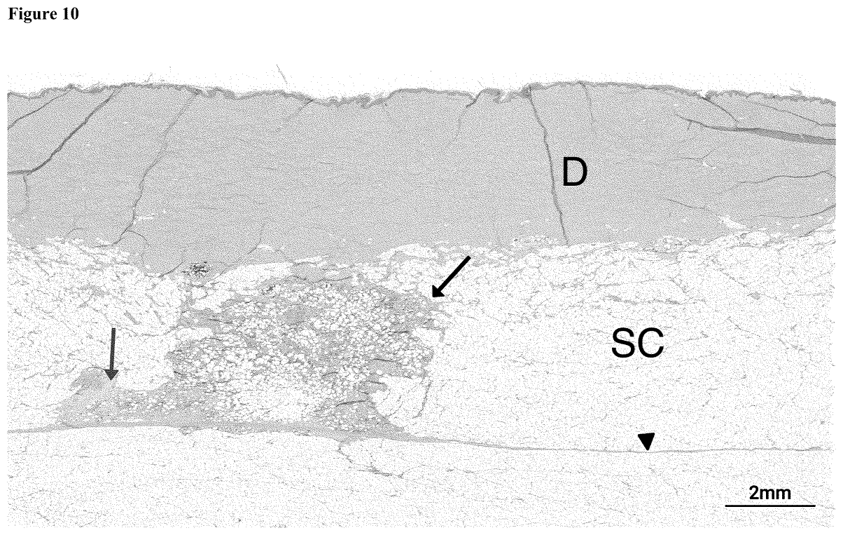

FIG. 10 shows Animal 1001, Area 4, Site A (NS siRNA Control, Equivalent to 200 μg/ml Dosage) Skin. H&E, 10×. Similar to what was seen in Area 3 (Image 2), there is slight narrowing of the superficial (above the fascia; black arrowhead) subcuticular adipose tissue (SC) with granulomatous inflammation (black arrow) containing an area of fibrosis (blue arrow). The dermis (D) is indicated.

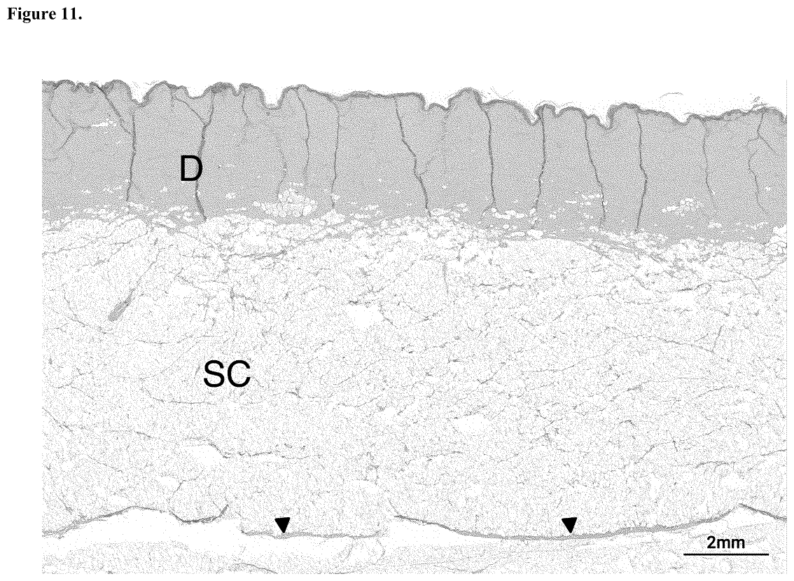

FIG. 11 shows Animal 2001, Untreated Area, Site 2 Skin. H&E, 10×. This region of untreated skin is histologically unremarkable. The dermis (D), subcuticular adipose (SC), and the most superficial fascial layer (black arrowheads) are indicated.

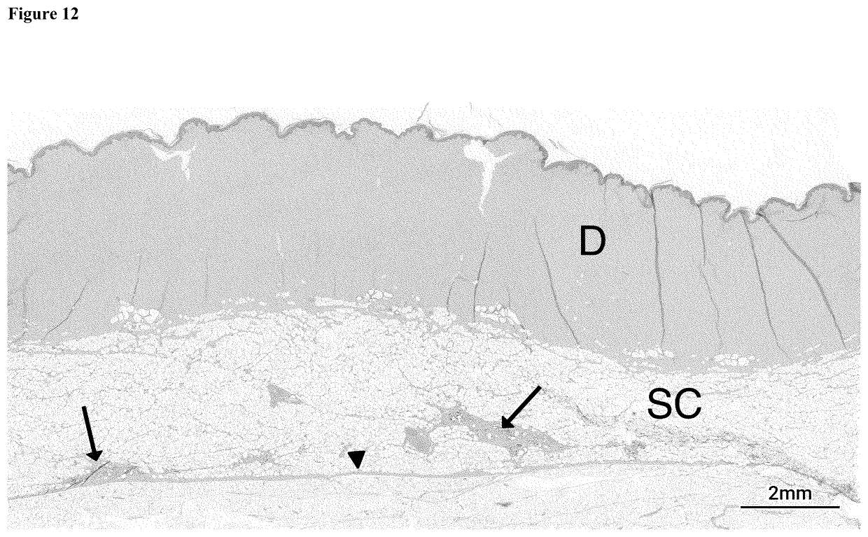

FIG. 12 shows Animal 2001, Area 3, Site A (TGF-β1 alone siRNA, 100 μg/ml), Skin. H&E, 10×. Areas of granulomatous inflammation (black arrows) surrounded by a small amount of increased collagen deposition/fibrosis are present within the superficial subcutis (SC). The superficial fascial layer (black arrowhead) and the dermis (D) are indicated.

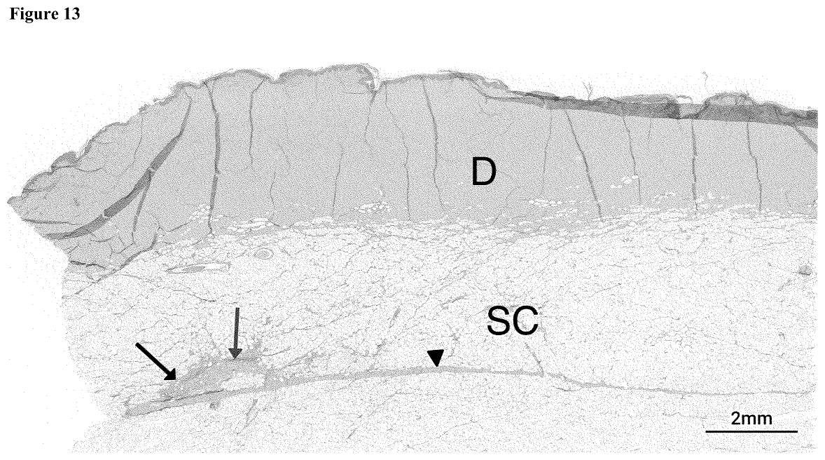

FIG. 13 shows Animal 2001, Area 4, Site A (NS siRNA Control, equivalent to 100 μg/ml Dosage) Skin. H&E, 10×. A small area of granulomatous inflammation (black arrow) with fibrosis (blue arrow) is visible within the superficial subcutis (SC), with relatively slight narrowing of this layer observed. The superficial fascia (black arrowhead) and the dermis (D) are indicated.

1. A method of remodeling adipose tissue of a patient comprising administering an effective amount of a nanoparticle formulation comprising at least one siRNA that binds to an mRNA that codes for transforming growth factor beta 1 (TGF-β1) and at least one siRNA that binds to an mRNA that codes for cyclooxygenase 2 (Cox2), or prostaglandin-endoperoxide synthase 2 (PTGS2), and a pharmaceutically acceptable carrier.

2. The method according to claim 1 wherein the nanoparticle formulation comprises a pharmaceutically acceptable histidine/lysine copolymer.

3. The method according to claim 2, wherein the copolymer is an HKP and/or HKP(+H).

4. The method according to claim 3, wherein said copolymer is H3K4b.

5. The method according to claim 1, wherein said formulation is administered through intradermal or subdermal injection.

6. The method according to claim 1, wherein said formulation is administered through intravenous (systemic) administration.

7. The method according to claim 1, wherein the siRNA comprises oligonucleotides between 19 and 27 nucleotides in length.

8. The method according to claim 5, wherein the siRNA comprises oligonucleotides at least 19 nucleotides in length.

9. The method according to claim 1, wherein said formulation is STP705.

10. The method according to claim 1, wherein said adipose tissue is selected from the group consisting of submental, abdominal, thigh (including inner and/or outer thigh), buttock, arm, and breast (including gynecomastia) adipose tissue.

11. A method of treating a patient with RNAi therapy by administering a nanoparticle formulation to remodel or to sculpt the patient's adipose tissue, comprising:
locally administering to the patient the nanoparticle formulation comprising:
one or more double stranded nucleic acids capable of inhibiting the activity of one or more target genes,
a pharmaceutically acceptable carrier, and
a polypeptide polymer comprising a histidine and a lysine residue,
wherein a first target gene encodes transforming growth factor beta 1 (TGF-β1), and
wherein a second target gene encodes cyclooxygenase 2 (Cox2) or prostaglandin-endoperoxide synthase 2 (PTGS2), and
wherein the adipose tissue is remodeled or sculpted in a patient suffering from metabolic disruption of adipose tissue.

12. The method according to claim 11 wherein the nanoparticle comprises HKP or HKP(+H).

13. The method according to claim 11 wherein the double stranded nucleic acid comprises a siRNA, shRNA or miRNA molecule.

14. The method according to claim 13 wherein the siRNA molecule comprises a conjugated molecule.

15. The method according to claim 11 wherein the one or more double stranded nucleic acids comprises oligonucleotides, each between 19 and 27 nucleotides in length.

16. The method according to claim 15 wherein the double stranded nucleic acid comprises oligonucleotides at least 19 nucleotides in length.

17. The method according to claim 11 wherein said adipose tissue is selected from the group consisting of submental, abdominal, thigh (including inner and/or outer thigh), buttock, arm, and breast (including gynecomastia) adipose tissue.

18. The method according to claim 10 wherein said formulation is STP705.

19. The method according to claim 5, wherein the siRNA is 25 nucleotides in length.

20. The method according to claim 15, wherein the siRNA is 25 nucleotides in length.


SEQUENCE LISTING (59 sequences)

SEQ ID NO: 1 - RNA, 25 nt, synthetic construct
  misc_feature 1..25: note = synthetic
  source 1..25: mol_type = other RNA
cccaagggctaccatgccaacttct

SEQ ID NO: 2 - RNA, 25 nt, synthetic construct
  misc_feature 1..25: note = synthetic
  source 1..25: mol_type = other RNA
agaagttggcatggtagcccttggg

SEQ ID NO: 3 - RNA, 25 nt, synthetic construct
  misc_feature 1..25: note = synthetic
  source 1..25: mol_type = other RNA
ggtctggtgcctggtctgatgatgt

SEQ ID NO: 4 - RNA, 25 nt, synthetic construct
  misc_feature 1..25: note = synthetic
  source 1..25: mol_type = other RNA
acatcatcagaccaggcaccagacc

SEQ ID NO: 5 - RNA, 25 nt, synthetic construct
  misc_feature 1..25: note = synthetic
  source 1..25: mol_type = other RNA
ggtggctggaacagccagatgtgtt

SEQ ID NO: 6 - RNA, 25 nt, synthetic construct
  misc_feature 1..25: note = synthetic
  source 1..25: mol_type = other RNA
aacacatctggctgttccagccacc

SEQ ID NO: 7 - RNA, 25 nt, synthetic construct
  misc_feature 1..25: note = synthetic
  source 1..25: mol_type = other RNA
gaggagccttcaggattacaagatt

SEQ ID NO: 8 - RNA, 25 nt, synthetic construct
  misc_feature 1..25: note = synthetic
  source 1..25: mol_type = other RNA
aatcttgtaatcctgaaggctcctc

SEQ ID NO: 9 - RNA, 25 nt, synthetic construct
  misc_feature 1..25: note = synthetic
  source 1..25: mol_type = other RNA
gctgaccctgaagttcatctgcatt

SEQ ID NO: 10 - RNA, 25 nt, synthetic construct
  misc_feature 1..25: note = synthetic
  source 1..25: mol_type = other RNA
aatgcagatgaacttcagggtcagc

SEQ ID NO: 11 - RNA, 25 nt, synthetic construct
  misc_feature 1..25: note = synthetic
  source 1..25: mol_type = other RNA
ggatccacgagcccaagggctacca

SEQ ID NO: 12 - RNA, 25 nt, synthetic construct
  misc_feature 1..25: note = synthetic
  source 1..25: mol_type = other RNA
tggtagcccttgggctcgtggatcc

SEQ ID NO: 13 - RNA, 25 nt, synthetic construct
  misc_feature 1..25: note = synthetic
  source 1..25: mol_type = other RNA
cccaagggctaccatgccaacttct

SEQ ID NO: 14 - RNA, 25 nt, synthetic construct
  misc_feature 1..25: note = synthetic
  source 1..25: mol_type = other RNA
agaagttggcatggtagcccttggg

SEQ ID NO: 15 - RNA, 25 nt, synthetic construct
  misc_feature 1..25: note = synthetic
  source 1..25: mol_type = other RNA
gagcccaagggctaccatgccaact

SEQ ID NO: 16 - RNA, 25 nt, synthetic construct
  misc_feature 1..25: note = synthetic
  source 1..25: mol_type = other RNA
agttggcatggtagcccttgggctc

SEQ ID NO: 17 - RNA, 25 nt, synthetic construct
  misc_feature 1..25: note = synthetic
  source 1..25: mol_type = other RNA
gatccacgagcccaagggctaccat

SEQ ID NO: 18 - RNA, 25 nt, synthetic construct
  misc_feature 1..25: note = synthetic
  source 1..25: mol_type = other RNA
atggtagcccttgggctcgtggatc

SEQ ID NO: 19 - RNA, 25 nt, synthetic construct
  misc_feature 1..25: note = synthetic
  source 1..25: mol_type = other RNA
cacgagcccaagggctaccatgcca

SEQ ID NO: 20 - RNA, 25 nt, synthetic construct
  misc_feature 1..25: note = synthetic
  source 1..25: mol_type = other RNA
tggcatggtagcccttgggctcgtg

SEQ ID NO: 21 - RNA, 25 nt, synthetic construct
  misc_feature 1..25: note = synthetic
  source 1..25: mol_type = other RNA
gaggtcacccgcgtgctaatggtgg

SEQ ID NO: 22 - RNA, 25 nt, synthetic construct
  misc_feature 1..25: note = synthetic
  source 1..25: mol_type = other RNA
ccaccattagcacgcgggtgacctc

SEQ ID NO: 23 - RNA, 25 nt, synthetic construct
  misc_feature 1..25: note = synthetic
  source 1..25: mol_type = other RNA
gtacaacagcacccgcgaccgggtg

SEQ ID NO: 24 - RNA, 25 nt, synthetic construct
  misc_feature 1..25: note = synthetic
  source 1..25: mol_type = other RNA
cacccggtcgcgggtgctgttgtac

SEQ ID NO: 25 - RNA, 25 nt, synthetic construct
  misc_feature 1..25: note = synthetic
  source 1..25: mol_type = other RNA
gtggatccacgagcccaagggctac

SEQ ID NO: 26 - RNA, 25 nt, synthetic construct
  misc_feature 1..25: note = synthetic
  source 1..25: mol_type = other RNA
gtagcccttgggctcgtggatccac

SEQ ID NO: 27 - RNA, 25 nt, synthetic construct
  misc_feature 1..25: note = synthetic
  source 1..25: mol_type = other RNA
ggtctggtgcctggtctgatgatgt

SEQ ID NO: 28 - RNA, 25 nt, synthetic construct
  misc_feature 1..25: note = synthetic
  source 1..25: mol_type = other RNA
acatcatcagaccaggcaccagacc

SEQ ID NO: 29 - RNA, 25 nt, synthetic construct
  misc_feature 1..25: note = synthetic
  source 1..25: mol_type = other RNA
gagcaccattctccttgaaaggact

SEQ ID NO: 30 - RNA, 25 nt, synthetic construct
  misc_feature 1..25: note = synthetic
  source 1..25: mol_type = other RNA
agtcctttcaaggagaatggtgctc

SEQ ID NO: 31 - RNA, 25 nt, synthetic construct
  misc_feature 1..25: note = synthetic
  source 1..25: mol_type = other RNA
cctcaattcagtctctcatctgcaa

SEQ ID NO: 32 - RNA, 25 nt, synthetic construct
  misc_feature 1..25: note = synthetic
  source 1..25: mol_type = other RNA
ttgcagatgagagactgaattgagg

SEQ ID NO: 33 - RNA, 25 nt, synthetic construct
  misc_feature 1..25: note = synthetic
  source 1..25: mol_type = other RNA
gatgtttgcattctttgcccagcac

SEQ ID NO: 34 - RNA, 25 nt, synthetic construct
  misc_feature 1..25: note = synthetic
  source 1..25: mol_type = other RNA
gtgctgggcaaagaatgcaaacatc

SEQ ID NO: 35 - RNA, 25 nt, synthetic construct
  misc_feature 1..25: note = synthetic
  source 1..25: mol_type = other RNA
gtctttggtctggtgcctggtctga

SEQ ID NO: 36 - RNA, 25 nt, synthetic construct
  misc_feature 1..25: note = synthetic
  source 1..25: mol_type = other RNA
tcagaccaggcaccagaccaaagac

SEQ ID NO: 37 - RNA, 25 nt, synthetic construct
  misc_feature 1..25: note = synthetic
  source 1..25: mol_type = other RNA
gtgcctggtctgatgatgtatgcca

SEQ ID NO: 38 - RNA, 25 nt, synthetic construct
  misc_feature 1..25: note = synthetic
  source 1..25: mol_type = other RNA
tggcatacatcatcagaccaggcac

SEQ ID NO: 39 - RNA, 25 nt, synthetic construct
  misc_feature 1..25: note = synthetic
  source 1..25: mol_type = other RNA
caccattctccttgaaaggacttat

SEQ ID NO: 40 - RNA, 25 nt, synthetic construct
  misc_feature 1..25: note = synthetic
  source 1..25: mol_type = other RNA
ataagtcctttcaaggagaatggtg

SEQ ID NO: 41 - RNA, 25 nt, synthetic construct
  misc_feature 1..25: note = synthetic
  source 1..25: mol_type = other RNA
caattcagtctctcatctgcaataa

SEQ ID NO: 42 - RNA, 25 nt, synthetic construct
  misc_feature 1..25: note = synthetic
  source 1..25: mol_type = other RNA
ttattgcagatgagagactgaattg

SEQ ID NO: 43 - RNA, 25 nt, synthetic construct
  misc_feature 1..25: note = synthetic
  source 1..25: mol_type = other RNA
ggtggctggaacagccagatgtgtt

SEQ ID NO: 44 - RNA, 25 nt, synthetic construct
  misc_feature 1..25: note = synthetic
  source 1..25: mol_type = other RNA
aacacatctggctgttccagccacc

SEQ ID NO: 45 - RNA, 25 nt, synthetic construct
  misc_feature 1..25: note = synthetic
  source 1..25: mol_type = other RNA
gctggaacagccagatgtgttgcca

SEQ ID NO: 46 - RNA, 25 nt, synthetic construct
  misc_feature 1..25: note = synthetic
  source 1..25: mol_type = other RNA
tggcaacacatctggctgttccagc

SEQ ID NO: 47 - RNA, 25 nt, synthetic construct
  misc_feature 1..25: note = synthetic
  source 1..25: mol_type = other RNA
cgccagattaccatctggtttcaga

SEQ ID NO: 48 - RNA, 25 nt, synthetic construct
  misc_feature 1..25: note = synthetic
  source 1..25: mol_type = other RNA
tctgaaaccagatggtaatctggcg

SEQ ID NO: 49 - RNA, 25 nt, synthetic construct
  misc_feature 1..25: note = synthetic
  source 1..25: mol_type = other RNA
ggagcccggcaattatgccaccttg

SEQ ID NO: 50 - RNA, 25 nt, synthetic construct
  misc_feature 1..25: note = synthetic
  source 1..25: mol_type = other RNA
caaggtggcataattgccgggctcc

SEQ ID NO: 51 - RNA, 25 nt, synthetic construct
  misc_feature 1..25: note = synthetic
  source 1..25: mol_type = other RNA
caaggatatcgaaggcttgctggga

SEQ ID NO: 52 - RNA, 25 nt, synthetic construct
  misc_feature 1..25: note = synthetic
  source 1..25: mol_type = other RNA
tcccagcaagccttcgatatccttg

SEQ ID NO: 53 - RNA, 25 nt, synthetic construct
  misc_feature 1..25: note = synthetic
  source 1..25: mol_type = other RNA
ggacaagaggcgcaagatctcggca

SEQ ID NO: 54 - RNA, 25 nt, synthetic construct
  misc_feature 1..25: note = synthetic
  source 1..25: mol_type = other RNA
tgccgagatcttgcgcctcttgtcc

SEQ ID NO: 55 - RNA, 25 nt, synthetic construct
  misc_feature 1..25: note = synthetic
  source 1..25: mol_type = other RNA
gcaagatctcggcagccaccagcct

SEQ ID NO: 56 - RNA, 25 nt, synthetic construct
  misc_feature 1..25: note = synthetic
  source 1..25: mol_type = other RNA
aggctggtggctgccgagatcttgc

SEQ ID NO: 57 - RNA, 25 nt, synthetic construct
  misc_feature 1..25: note = synthetic
  source 1..25: mol_type = other RNA
ccatctggtttcagaaccgccgggt

SEQ ID NO: 58 - RNA, 25 nt, synthetic construct
  misc_feature 1..25: note = synthetic
  source 1..25: mol_type = other RNA
acccggcggttctgaaaccagatgg

SEQ ID NO: 59 - RNA, 25 nt, unidentified
  source 1..25: mol_type = genomic RNA; note = the only information available for the source of this sequence is that it is taken from pig genome; no knowledge of species was provided
cccaagggctaccatgccaatttct